(19) United States
(12) Patent Application Publication
(10) Pub. No.: US 2024/0174430 A1
(43) Pub. Date: May 30, 2024

(54) Finishing Rinse Dispenser

(71) Applicant: James Bradford, Scottsdale, AZ (US)

(72) Inventor: James Bradford, Scottsdale, AZ (US)

(21) Appl. No.: 18/070,795

(22) Filed: Nov. 29, 2022

(51) Int. Cl. B65D 83/38; B65D 83/66

(52) U.S. Cl. CPC B65D 83/38; B65D 83/666; B65D 83/30

(57) ABSTRACT

A pressure vessel apparatus for dispensing skin care products comprises a multi-chamber bottle capable of containing a pressure of above 35 psi. Separate chambers for liquid contents, mixing of the liquid with active ingredients, and effectively dispensing the resulting product are provided.

### TECHNICAL FIELD OF THE INVENTION

The present disclosure relates to an apparatus for delivering a fluid, such as a moisturizing agent to a surface such as the body of the user.

### BACKGROUND OF THE INVENTION

Many individuals benefit from the application of a skin treatment as a final rinse. This may be for instance either after showering or getting out of the water after swimming. A softener, pH neutralizer or lotion may be desired. A dermatological patient may use a sterilized solution. In each case, it is desirable to employ a reusable handheld container that can dispense pressurized contents that the user can easily apply to the entire body.

Pressurization of the contents may preferably be achieved by the mixture of water with an activating agent that produces a gas evolution reaction. A vessel configuration that keeps the activating agent separate from the water or other liquid, is easy to fill and store, and can be readily manipulated to induce the gas evolution reaction is desirable. The dispenser should be easy to hold in one hand and direct the contents over the entire body.

### SUMMARY OF THE INVENTION

A pressure vessel apparatus for dispensing skin care products comprises a multi-chamber bottle capable of containing an internal pressure of about 35 to 60 psi. In an embodiment, a larger lower chamber holds a carrier liquid, such as water. Above the lower chamber is a smaller mixing chamber that will retain an activation compound so that the compound does not touch the carrier liquid. An upper dispensing chamber has apertures that allow the contents to exit the vessel.

In use, the lower chamber is filled with a carrier liquid, such as water. An activation compound, usually a dry substance, is placed in a retainer in the mixing chamber. The vessel is kept in a vertical orientation, so the liquid and the activation compound do not interact. After completing a shower or exiting a body of water, the user inverts the vessel so the carrier liquid envelops the activation compound, triggering a gas evolution reaction and generating a pressurized mixture of liquid and skin treatment ingredients. The pressure expels the mixture through the dispensing apertures and the user directs the flow onto the body.

### BRIEF DESCRIPTION OF THE DRAWINGS

FIG. 1 is a depiction of an embodiment of the current invention.

FIG. 2 is a cutaway view of the inside of the embodiment of FIG. 1.

FIG. 3 is a detailed view of the storage pocket of an activation chamber in an embodiment of the current invention.

FIG. 4A shows a cutaway view of a portion of an embodiment of the current invention having a valved nozzle in the open position.

FIG. 4B shows the same component with the nozzle in the closed position.

FIG. 5 shows a cutaway view of a portion of an alternative embodiment of the invention.

### DETAILED DESCRIPTION

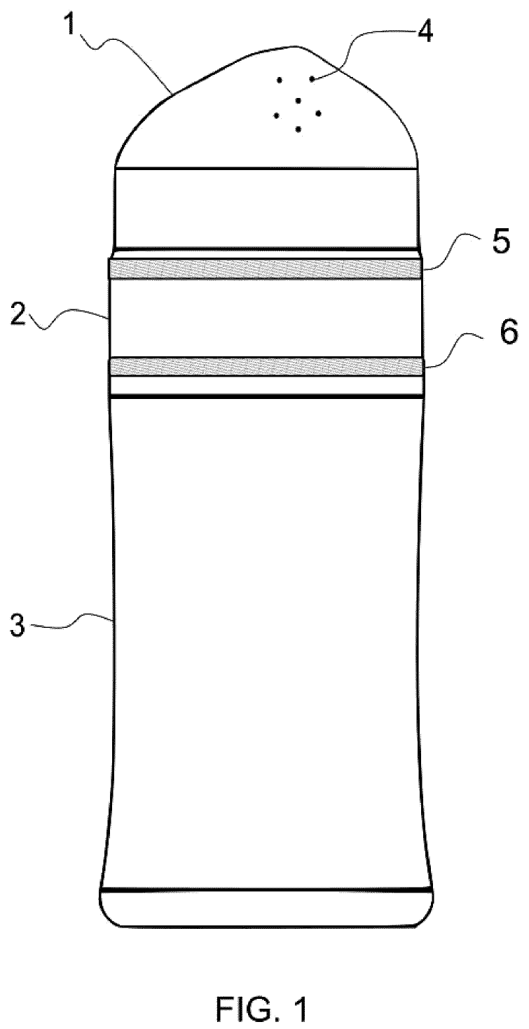

FIG. 1 depicts an embodiment of a vessel according to the current invention. Liquid container chamber 3 is for holding the liquid that will be the carrier for the skin treatment preparation. Activation chamber 2 sits above liquid container chamber 3 and may be secured by matching screw threads. Upper dispensing chamber 1 sits above activation chamber 2 and is also secured by screw threads. External gaskets 5 and 6 provide a liquid tight seal, rated at about 40 psi or more, where mixing chamber 2 joins lower chamber 3 and upper dispensing chamber 1.

The vessel may conventionally be generally cylindrical and dimensioned to hold in one hand and contain about a half-liter of liquid. Suitable materials that are lightweight and capable of sustaining internal pressures of 35 psi to 60 psi include High Density Polyethylene (HDPE), Low Density Polyethylene (LDPE), Polypropylene (PP), Polyvinyl Chloride (PVC), Polyethylene Terephthalate (PET), Thermoplastic Elastomers (TPE) and Acrylonitrile Butadiene Styrene (ABS). There may be applications in which stronger, heavier materials are used.

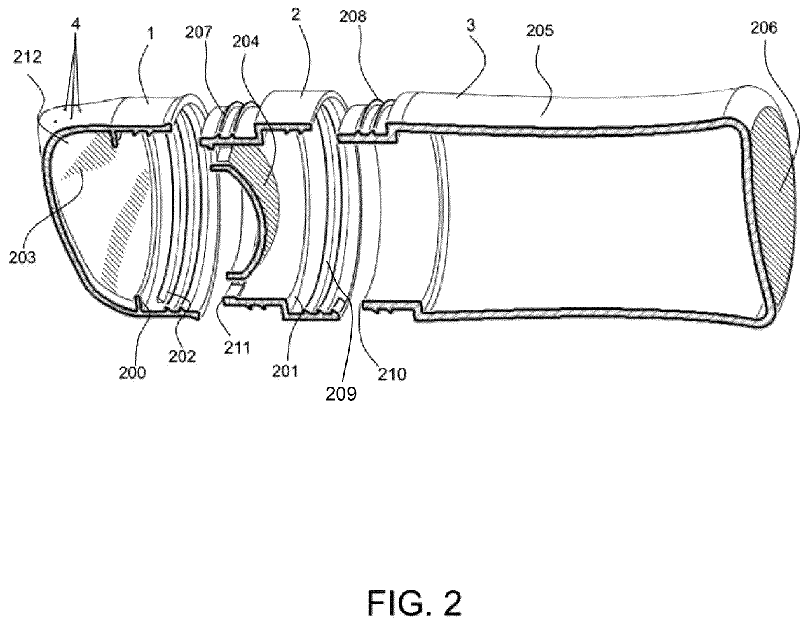

FIG. 2 shows the internal configuration of an embodiment of a vessel. Liquid container chamber 3 has an inwardly curved wall 205 similar to an hourglass or slim waist bottle. This contour strengthens the wall against interior pressure generated by activation of the contents and makes the vessel easier to grip. The bottom 206 is preferably convex, affording added pressure resistance and a stable platform for standing the vessel on a shelf or other surface. The open upper end 210 is threaded. In a preferred embodiment, buttress threads 208 are incorporated to resist the internal pressure that tends to separate the lower chamber from the vessel. A large thread pitch is preferably employed to minimize the number of rotations required to assemble the vessel in use.

Cylindrical activation chamber 2 incorporates buttress threads 209 corresponding to the threads 208 in the lower chamber. An internal pressure gasket seat 201 is secured against the open end 210 of the lower chamber 3 when the parts are screwed together. This further resists leakage of pressurized contents at the joint between the chambers. The upper end 211 of activation chamber 2 incorporates buttress threads 207 similar to those 208 on the lower chamber 3. Activator storage element 204 is configured to hold an activation compound and keep the compound from contact with the carrier liquid in lower chamber 3 until the user initiates activation.

Dispensing chamber 1 is the lid of the vessel and is configured to disperse the pressurized activated contents as directed by the user. Buttress threads 202 correspond to the threads 207 in the activation chamber 2. An internal pressure gasket seat 200 is secured against the upper end 211 of activation chamber 2 when the parts are screwed together. In the illustrated embodiment, the inner configuration 203 of the dispensing chamber 1 funnels all contents to a low point 212 where exit apertures 4 are located when the vessel is inverted for use.

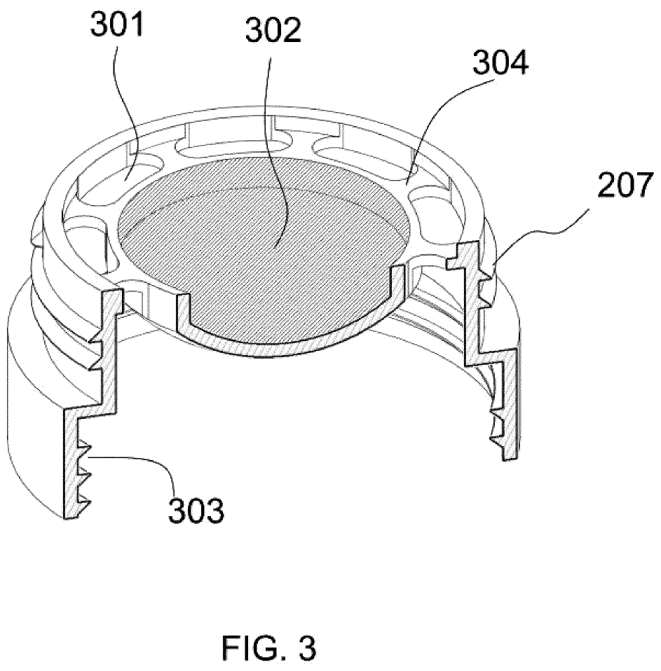

An embodiment of activation chamber 2 is shown in FIG. 3. An activator storage element comprising a concave containment pocket 203 is supplied to hold an activator compound and keep it from contact with the carrier liquid in lower chamber 3 while the vessel is right-side up. A flange 304 connecting the containment pocket to the wall of the activation chamber incorporates a plurality of apertures 301. These apertures will allow the carrier liquid to flood and interact with the activator compound when the vessel is inverted for active use. This view also shows the buttress threads 207 at the upper end of activation chamber 2, the buttress threads 303 that engage the threads 208 of the lower chamber 3 and the internal pressure gasket seat 201. It should be noted that although this component is designated the activation chamber, the mixing of the carrier liquid and the activation compound and the contemporaneous gas generation, once begun, are not limited to this chamber, but instead extend throughout the vessel.

Use of the disclosed embodiment is simple when, for example, applying a finishing body rinse after a shower. The user unscrews the top and middle chambers and fills the bottom chamber with water. The activation chamber is attached to the liquid container chamber and a dry activation compound is placed in the pocket of the activator storage element, after which the dispensing chamber is screwed onto the activation chamber. At all times the vessel is maintained in a vertical orientation so the water (or other liquid) and the activation compound do not interact. The vessel may be set on a shelf, sill or other surface near the shower. When ready, the user inverts the vessel so the water passes through the apertures in the containment element and floods the activation compound, triggering a gas evolution reaction that rapidly mixes the ingredients and generates internal pressure in the vessel. In some instances, the vessel may be shaken vigorously to accelerate the reaction. The contents are expelled under pressure through the exit apertures as a spray, which the user, holding the inverted vessel, may direct to all parts of the body. Depending on the ingredients, it may take 30 to 60 seconds to spray out all the contents. The vessel may then be disassembled and rinsed out for later reuse.

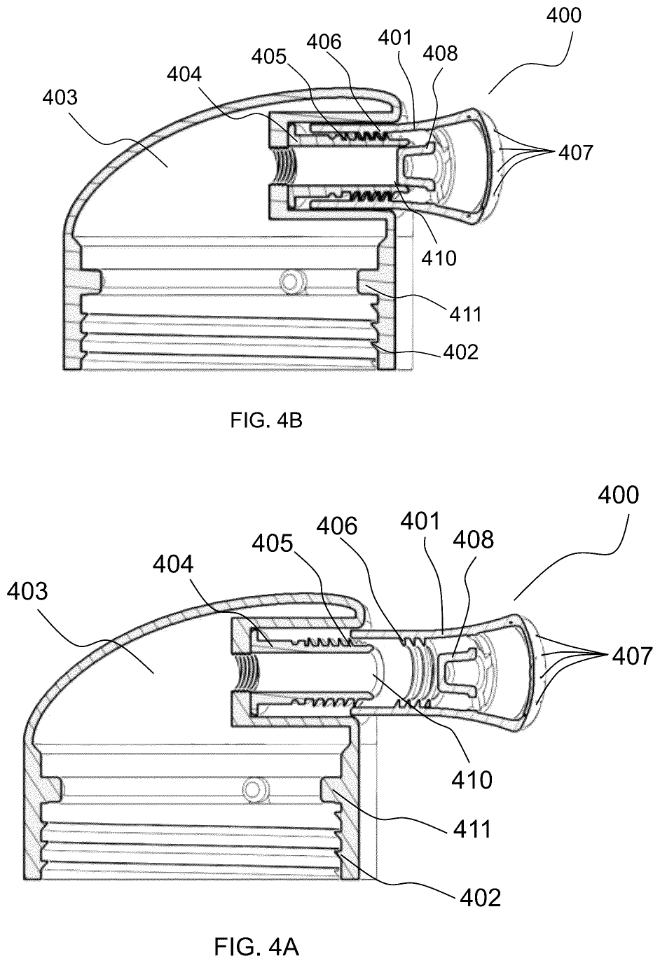

An alternative embodiment of a dispensing chamber, having a closeable spray nozzle 400, is shown in FIGS. 4A and 4B. Buttress threads 402 correspond to the threads 207 in the mixing chamber 2. An internal pressure gasket seat 411 is secured against the upper end 211 of mixing chamber 2 when the parts are screwed together. Valve stem 404 with external threads 405 extends into the interior 403 of the dispensing chamber. A spray nozzle tube 401 with internal threads 406 corresponding to the valve stem threads 405 includes a plurality of exit apertures 407 and an integrated stopper 408 dimensioned to block the open end 410 of the valve stem 404. When the nozzle tube is extended outward, as in FIG. 4A, internal pressurized contents will flow through the valve stem 404, around the stopper 408 and out through the exit apertures 407 for distribution onto the user's body. The spray nozzle may be closed by twisting it, usually clockwise, so that the threads of the valve stem interacting with the threads of the nozzle tube pull the nozzle and the stopper inward, whereupon the stopper closes the open end of the valve stem, as seen in FIG. 4B.

In use, the valve will start in the closed position while the carrier liquid remains in the lower chamber and the activation compound is inert. Once the vessel is inverted to initiate a gas evolution reaction, the user may wait a few seconds to allow pressure to build throughout the vessel, then twist the nozzle to disengage the stopper and begin spraying the pressurized contents. Depending on the vessel construction and the nature of the specific gas evolution reaction induced, it may be necessary to take precautions against excessive pressure buildup, which could lead to structural damage or a high-pressure dispensing spray that is harmful. In the disclosed embodiment, the stopper could be designed so that it does not fully block the outward flow.

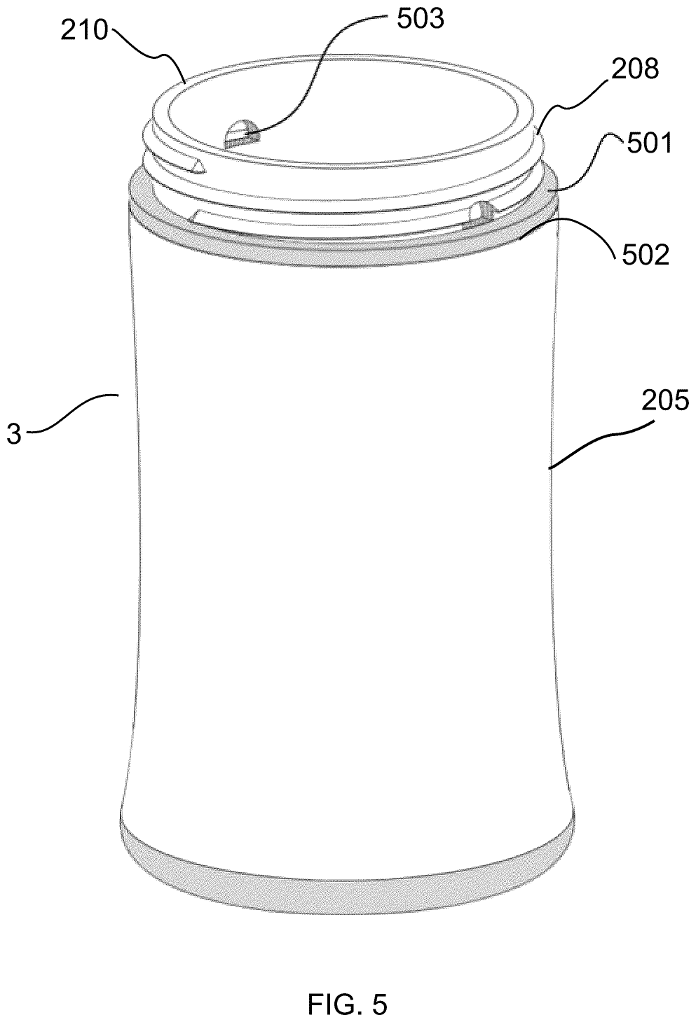

In another embodiment, pressure resistance of the vessel may be enhanced, as seen in FIG. 5, by adding a compressible gasket 502 to the shoulder 502 formed where the vessel wall 205 meets the threaded section 208 of liquid container chamber 3. When the liquid container chamber 3 is tightened against the activation chamber 2, by for example screwing them together, gasket 502 is compressed, forming a pressure seal at the outside walls of the two chambers. A similar gasket may be placed on a corresponding shoulder on the activation chamber 2, forming a pressure seal at the connection between activation chamber 2 and dispensing chamber 1. This configuration will also accommodate optional weep holes 503 in the sides of the liquid containment chamber above the gasket 502, preventing overfilling of the chamber.

A variety of activation compounds may be employed with the finishing rinse dispenser, and users may choose to formulate their own by compressing ingredients into a tablet form or by encapsulating in a polyvinyl alcohol packet similar to a dishwasher or laundry detergent pod. Although there are many compounds that will generate a gas evolution reaction in the presence of water, a simple and safe option comprises sodium bicarbonate and citric acid in appropriate proportions. These may be combined into a dry pellet with skin treatment ingredients. Many commercially available “bath bombs” for bathing use rely on this chemistry, and although the generally popular bath bomb products are too large to fit into the mixing chamber carrier of the preferred embodiment, one could slice off a segment of a bath bomb and use it with the invention.

Experimentation has shown that an effective activation compound which provides desirable skin treatment and generates sufficient gas pressure for effective dispensing comprises 65 g sodium bicarbonate, 35 g citric acid, 50 g magnesium sulfate, 25 g starch (corn/rice) and 1 g borax. An effective formulation for dechlorinating the skin after swimming is 65 g sodium bicarbonate, 35 g citric acid, 50 g magnesium sulfate, 15 g starch, 30 g ascorbic acid and 1 g borax.

Generic Formulations used for bath bombs/bath salts incorporate the same composition for gas evolution initiation. Chosen formulations can have skin softening oils (wet agents) added to the salts formula as long as the skin care agent is an anhydrous compound or product. Preferably the result of mixing the carrier fluid with the soda acid chemistry will create a skin care emulsification solution and produce enough gas to expel or spray out the solution, under high pressure, for a long enough time, that the user can deliver the freshly created skin care solution to the entirety of their body themselves.

Many skin care oils or possibly any anhydrous skin care therapeutic or emollient compound can be combined with the dry gas producing soda acid powder mix without impeding the gas reaction of the soda acid chemistry. This effectively enables the user to instantly make a watered-down solution of skin care lotion that can be sprayed onto the skin as a final rinse after showering. In the case of skin oils for application, because oil and water do not mix, the oil will separate without sufficient agitation; suitable chemistries known in the art, such as a mild surfactant, may be employed so that after activation the oil is substantially uniformly distributed throughout the liquid phase of the bottle.

In the process of making the formula into a hard tablet, the dry agents are mixed and water is added in a gentle fashion by spray bottle or misting into the dry powder so the reaction does not start to “fizz” but will introduce just enough hydration to allow the powder to be packed into a ball or tablet. The addition of oils intended to moisturize the skin or other anhydrous agents such as surfactants that aid in the emulsification of the skin care agents into solution may be accomplished without starting any kind of gas producing reaction with the dry powder.

In some cases, the desired skin treatment, in the form of an oil or a lotion, may be poured intact into the carrier chamber for eventual dispensing. The dry activator compound in the mixing chamber may be one that initiates a gas evolution reaction but contains no skin treatment ingredients, so that it serves only to assist in the even and efficient dispensing of the active ingredients.

The foregoing description has been presented and is intended for the purposes of illustration and description. It is not intended to be exhaustive nor limit the invention to the above teachings. The embodiments were chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

Numerous other changes, substitutions, variations, alterations, and modifications may be ascertained to one skilled in the art and it is intended that the present invention encompass all such changes, substitutions, variations, alterations, and modifications as falling within the spirit and scope of the appended claims.

1. A multi-chamber pressure vessel for dispensing a skin treatment solution comprising:
a generally cylindrical activation chamber open at a first and a second end; an activator storage element attached to the activation chamber by a structure with apertures that allow passage of liquid entering the activation chamber from its first end;
a liquid container chamber removably attached to the first end of the activation chamber, comprising a generally cylindrical pressure vessel with an open end and a closed bottom;
and a dispensing chamber, removably attachable to the second end of the activation chamber, having an open end and a closed end with at least one exit aperture; wherein
the chambers are capable of containing internal pressures of at least 35 psi and the chambers are attachable with connections that stop leakage of liquid contents under at least 35 psi of pressure.

2. The vessel of claim 1 wherein the open end of the liquid container chamber and the first end of the activation chamber have matching abutment threads and the activation chamber has an internal pressure gasket seat to engage the open end of the liquid container chamber.

3. The vessel of claim 2 wherein the open end of the dispensing chamber and the second end of the activation chamber have matching abutment threads and the dispensing chamber has an internal pressure gasket seat to engage the second end of the activation chamber.

4. The vessel of claim 1 wherein the open end of the dispensing chamber and the second end of the activation chamber have matching abutment threads and the dispensing chamber has an internal pressure gasket seat to engage the second end of the activation chamber.

5. The vessel of claim 3 wherein the liquid container chamber has an inwardly convex closed end and an inwardly curved interior wall.

6. The vessel of claim 3 wherein the activator storage element comprises an open pocket attached to the activation chamber by a flange having spaced apart apertures.

7. The vessel of claim 5 wherein the liquid container chamber holds about one-half liter of liquid.

8. The vessel of claim 6 further including an external compressible gasket on a shoulder where the liquid container chamber and the activation chamber meet and an external compressible gasket on a shoulder where the activation chamber and the dispensing chamber meet.

9. The vessel of claim 6 wherein the shape of the dispensing chamber channels liquid to a low point with a plurality of exit apertures directed approximately perpendicular to the vessel wall when the vessel is inverted.

10. The vessel of claim 6 wherein the dispensing chamber includes a closeable spray nozzle that may be set to an open position and to a closed position.

11. The vessel of claim 1 wherein the open end of the liquid container chamber and the first end of the activation chamber have matching abutment threads and the activation chamber has an internal pressure gasket seat to engage the open end of the liquid container chamber; the open end of the dispensing chamber and the second end of the activation chamber have matching abutment threads and the dispensing chamber has an internal pressure gasket seat to engage the second end of the activation chamber; the activator storage element comprises an open pocket attached to the activation chamber by a flange having spaced apart apertures; the liquid container chamber has an inwardly convex closed end and an inwardly curved interior wall and holds about one-half liter of liquid; and the shape of the dispensing chamber channels liquid to a low point with a plurality of exit apertures directed approximately perpendicular to the vessel wall when the vessel is inverted.

12. A method of applying a skin treatment solution to a body comprising the steps of:
inserting a gas evolution generating activation compound into a storage location of a multi-chamber pressure vessel for dispensing a skin treatment solution comprising a generally cylindrical activation chamber open at a first and a second end; an activator storage element comprising an open pocket attached to the activation chamber by a structure with apertures that allow passage of liquid entering the activation chamber from its first end; a liquid container chamber removably attached to the first end of the activation chamber, comprising a generally cylindrical pressure vessel with an open end and a closed bottom; and a dispensing chamber, removably attachable to the second end of the activation chamber, having an open end and a closed end with at least one exit aperture;
wherein the chambers are capable of containing internal pressures of at least 35 psi and the chambers are attachable with connections that stop leakage of liquid contents under at least 35 psi of pressure;
adding a carrier liquid to the liquid container chamber of the vessel;
attaching the liquid container chamber to the activation chamber of the vessel while maintaining the vessel in an upright position;
attaching the dispensing chamber to the activation chamber while continuing to maintain the vessel in an upright position;
inverting the vessel so the carrier liquid enters the activation chamber and contacts the activation compound, causing internal pressure to disperse a spray of skin treatment solution;
and directing the spray to desired parts of the body.

13. The method of claim 10, including the further step of shaking the vessel after inverting it, to accelerate the gas evolution and mixing of ingredients.

14. The method of claim 10 wherein the exit aperture comprises a closeable nozzle, including the steps of closing the nozzle before inverting the vessel and opening the nozzle after the vessel is inverted and internal pressure begins to build.
